(19) United States
(12) Patent Application Publication
(10) Pub. No.: US 2021/0227852 A1
(43) Pub. Date: Jul. 29, 2021

(54) PET FOOD COMPOSITIONS

(71) Applicant: HILL'S PET NUTRITION, INC., Topeka, KS (US)

(72) Inventors: Matthew JACKSON, Topeka, KS (US); Dennis JEWELL, Lawrence, KS (US)

(73) Assignee: HILL'S PET NUTRITION, INC., Topeka, KS (US)

(21) Appl. No.: 16/314,490

(22) PCT Filed: Dec. 17, 2018
(86) PCT No.: PCT/US2018/065978

§ 371 Date: Dec. 31, 2018

(51) Int. Cl. A23K 20/163; A23K 10/30; A23K 50/42; A61K 36/52; A61K 47/46; A61P 25/22

(52) U.S. Cl. CPC A23K 20/163; A23K 10/30; A61P 25/22; A61K 36/52; A61K 47/46; A23K 50/42

(57) ABSTRACT

Described herein are pet food compositions comprising: an insoluble fiber source comprising a lignin; a polyphenol source; and a soluble fiber source; wherein the weight ratio of insoluble fiber to soluble fiber is greater than about 2:1. Methods of making and using these compositions are also described.

### CROSS-REFERENCE TO RELATED APPLICATION

This application is a national stage entry under 35 U.S.C. § 371 of International Patent Application No. PCT/US2018/65978, filed Dec. 17, 2018, the contents of which are hereby incorporated herein by reference in their entirety.

### BACKGROUND

Pets are prone to food intake disruptions when schedules or environments vary due to pet owner's activities or travel (e.g. trips to the veterinarian). Decreased intake of nutritious foods can exacerbate anxiety and behavioral problems in pets experiencing these schedule variations. Despite advances in the area, there are limited options for pet parents who are trying to better harmonize their pet's schedules and intakes in an increasingly variable world; and for pets who need better nutrition to support their hectic lives.

Another dietary concern associated with schedule variation lies in the extent to which food is digested. For instance, there may be situations in which an animal exposed to certain stressors resumes its normal intake or increases its intake, but is unable to properly digest the food that is consumed.

As such, there remains a need for nutritional therapeutics which are capable of not only encouraging/stimulating food intake, but also ensure that the food consumed is properly digested. Ideally, the higher intakes could be maintained even in the face of schedule variations; but at a minimum, the animal would return to normal intake levels as soon as possible after the schedule disruption subsides.

Certain embodiments of the present invention are designed to address these, and other, needs.

### BRIEF SUMMARY

Some embodiments of the present invention provide a pet food composition comprising: an insoluble fiber source comprising a lignin; a polyphenol source; and a soluble fiber source; wherein the weight ratio of insoluble fiber to soluble fiber is greater than about 2:1.

Other embodiments provide compositions for stimulating food intake in a companion animal in need thereof, comprising: a source of soluble fiber; a complex comprising: a source of polyphenols; and mixture of at least two different sources of insoluble fiber; wherein the total dietary fiber content of the composition is greater than 5 percent on a dry matter basis (DMB).

Further embodiments provide composition for targeted modulation of the endocannabinoid system of a companion animal, comprising: a polyphenol source comprising a phenolic compound selected from ellagic acid; gallic acid; protocatechuic acid; p-hydroxybenzoic acid; catechin; and a combination of two or more thereof; and a fiber source comprising an insoluble fiber selected from: a lignin; tomato pomace; a natural vegetable food fiber; and a combination of two or more thereof.

Yet other embodiments provide methods for: a) increasing the food intake of a companion animal suffering from stress; b) maintaining the food intake of a companion animal during a stressful experience; c) increasing and/or maintaining the food intake of a companion animal suffering from anxiety; d) normalizing the food intake of a companion animal during schedule variations; e) decreasing fecal ethanolamides in a companion animal; f) decreasing fecal acyltaurines in a companion animal; g) decreasing fecal acylglycines in a companion animal; h) decreasing dietary protein putrefaction; i) increasing the weight of a companion animal suffering from anxiety; j) maintaining levels of acylglycerols; and/or k) modulating the endocannabinoid system of a companion animal, comprising: administering a composition comprising an insoluble fiber source comprising: a lignin; a polyphenol source; and a soluble fiber source; wherein the weight ratio of insoluble fiber to soluble fiber is greater than about 2:1, to a companion animal in need thereof.

### BRIEF DESCRIPTION OF THE DRAWINGS

FIG. 1 depicts data demonstrating the overall increased intake and recovery aptitude provided by compositions of the present invention in the face of schedule variations/disruptions.

FIG. 2 depicts data demonstrating the impact that an exemplary composition of the present invention has on ethanolamides, as compared to control composition.

FIG. 3 depicts data demonstrating the impact that an exemplary composition of the present invention has on taurines, as compared to a control composition.

FIG. 4 depicts data demonstrating the impact that an exemplary composition of the present invention has on glycines, as compared to a control composition.

FIG. 5 depicts data demonstrating the impact that an exemplary composition of the present invention has on glycerols, as compared to a control composition.

FIG. 6 depicts data demonstrating the impact that an exemplary composition of the present invention has on fecal and serum polyamines, as compared to a control composition.

FIG. 7 depicts data demonstrating the impact that an exemplary composition of the present invention has on fecal branched short chain fatty acids (BSCFA), as compared to a control composition.

### DETAILED DESCRIPTION

In some embodiments, the present invention provides a pet food composition comprising: an insoluble fiber source comprising a lignin; a polyphenol source; and a soluble fiber source; wherein the weight ratio of insoluble fiber to soluble fiber is greater than about 2:1.

In some embodiments, the polyphenol source comprises a mixture of phenolic compounds. In some embodiments, the polyphenol source comprises a phenolic compound selected from ellagic acid; gallic acid; protocatechuic acid; p-hydroxybenzoic acid; catechin; and a combination of two or more thereof. In some embodiments, the polyphenol source comprises pecan shells, or any other component of the pecan nut. In some embodiments, the pecan shell may also be a source of lignin-based fiber.

In some embodiments, the insoluble fiber source comprises a fiber bundle. In some embodiments, the fiber bundle may be referred to as a fiber triangle. In some embodiments, the fiber bundle comprises a mixture of at least two different sources of insoluble fiber. In some embodiments, the fiber bundle comprises a cellulosic material; tomato pomace and a natural vegetable food fiber.

In certain embodiments, the natural vegetable food fiber is selected from: carrot fiber; beet fiber; pea fiber; broccoli fiber; collard green fiber; swiss chard fiber; artichoke fiber; and a combination of two or more thereof. In some embodiments, the natural vegetable food fiber comprises pea fiber.

In some embodiments, the weight ratio of insoluble fiber to soluble fiber is greater than about 1.75:1. In some embodiments, the weight ratio of insoluble fiber to soluble fiber is greater than about 2.25:1. In some embodiments, the weight ratio of insoluble fiber to soluble fiber is greater than about 2.5:1. In some embodiments, the weight ratio of insoluble fiber to soluble fiber is greater than about 2.75:1.

In some embodiments, the weight ratio of insoluble fiber to soluble fiber is greater than about 3:1. In some embodiments, the weight ratio of insoluble fiber to soluble fiber is greater than about 3.25:1. In some embodiments, the weight ratio of insoluble fiber to soluble fiber is greater than about 3.5:1. In some embodiments, the weight ratio of insoluble fiber to soluble fiber is greater than about 3.75:1.

In some embodiments, the weight ratio of insoluble fiber to soluble fiber is greater than about 4:1. In some embodiments, the weight ratio of insoluble fiber to soluble fiber is greater than about 4.25:1. In some embodiments, the weight ratio of insoluble fiber to soluble fiber is greater than about 4.5:1. In some embodiments, the weight ratio of insoluble fiber to soluble fiber is greater than about 4.75:1.

In some embodiments, the weight ratio of insoluble fiber to soluble fiber is greater than about 5:1. In some embodiments, the weight ratio of insoluble fiber to soluble fiber is greater than about 5.25:1. In some embodiments, the weight ratio of insoluble fiber to soluble fiber is greater than about 5.5:1. In some embodiments, the weight ratio of insoluble fiber to soluble fiber is greater than about 5.75:1.

In some embodiments, the weight ratio of insoluble fiber to soluble fiber is greater than about 6:1. In some embodiments, the weight ratio of insoluble fiber to soluble fiber is greater than about 6.25:1. In some embodiments, the weight ratio of insoluble fiber to soluble fiber is greater than about 6.5:1. In some embodiments, the weight ratio of insoluble fiber to soluble fiber is greater than about 6.75:1.

In some embodiments, the weight ratio of insoluble fiber to soluble fiber is greater than about 7:1. In some embodiments, the weight ratio of insoluble fiber to soluble fiber is greater than about 7.25:1. In some embodiments, the weight ratio of insoluble fiber to soluble fiber is greater than about 7.5:1. In some embodiments, the weight ratio of insoluble fiber to soluble fiber is greater than about 7.75:1.

In some embodiments, the weight ratio of insoluble fiber to soluble fiber is greater than about 8:1. In some embodiments, the weight ratio of insoluble fiber to soluble fiber is greater than about 8.25:1. In some embodiments, the weight ratio of insoluble fiber to soluble fiber is greater than about 8.5:1. In some embodiments, the weight ratio of insoluble fiber to soluble fiber is greater than about 8.75:1.

In some embodiments, the weight ratio of insoluble fiber to soluble fiber is greater than about 9:1. In some embodiments, the weight ratio of insoluble fiber to soluble fiber is greater than about 9.25:1. In some embodiments, the weight ratio of insoluble fiber to soluble fiber is greater than about 9.5:1. In some embodiments, the weight ratio of insoluble fiber to soluble fiber is greater than about 9.75:1.

In some embodiments, the weight ratio of insoluble fiber to soluble fiber is greater than about 10:1. In some embodiments, the weight ratio of insoluble fiber to soluble fiber is about 10:1.

Still further embodiments provide compositions for stimulating food intake in a companion animal in need thereof, comprising: a source of soluble fiber; a complex comprising: a source of polyphenols; and a source of insoluble fiber comprising a fiber bundle; wherein the total dietary fiber content of the composition is greater than 5 percent on a dry matter basis (DMB).

In some embodiments, the compositions comprise less than about 1%, by weight, of soluble fiber. In some embodiments, the compositions comprise less than about 0.9%, by weight, of soluble fiber. In some embodiments, the compositions comprise less than about 0.8%, by weight, of soluble fiber. In some embodiments, the compositions comprise less than about 0.7%, by weight, of soluble fiber. In some embodiments, the compositions comprise less than about 0.6%, by weight, of soluble fiber. In some embodiments, the compositions comprise less than about 0.5%, by weight, of soluble fiber. In some embodiments, the compositions are substantially free of soluble fiber.

In some embodiments, the fiber bundle comprises from about 5% to about 15%, by weight, of the total composition. In some embodiments, the fiber bundle comprises about 5%, by weight, of the total composition. In some embodiments, the fiber bundle comprises about 6%, by weight, of the total composition. In some embodiments, the fiber bundle comprises about 7%, by weight, of the total composition. In some embodiments, the fiber bundle comprises about 8%, by weight, of the total composition. In some embodiments, the fiber bundle comprises about 9%, by weight, of the total composition. In some embodiments, the fiber bundle comprises about 10%, by weight, of the total composition. In some embodiments, the fiber bundle comprises about 11%, by weight, of the total composition. In some embodiments, the fiber bundle comprises about 12%, by weight, of the total composition. In some embodiments, the fiber bundle comprises about 13%, by weight, of the total composition. In some embodiments, the fiber bundle comprises about 14%, by weight, of the total composition. In some embodiments, the fiber bundle comprises about 15%, by weight, of the total composition.

In some embodiments, food intake is calculated based upon intake of calories normalized to metabolic bodyweight.

Further embodiments provide compositions for targeted modulation of the endocannabinoid system of a companion animal, comprising: a polyphenol source comprising a phenolic compound selected from ellagic acid; gallic acid; protocatechuic acid; p-hydroxybenzoic acid; catechin; and a combination of two or more thereof; and a fiber source comprising an insoluble fiber selected from: a lignin; tomato pomace; a natural vegetable food fiber; and a combination of two or more thereof.

In some embodiments, the composition is in the form of a kibble. In further embodiments, the composition is in the form of a multi-layer kibble. Yet other embodiments provide a multi-layer kibble comprising a coating. In some embodiments, the coating comprises a palatant. In some embodiments, the kibble is formed by extrusion.

In some embodiments, the kibble further comprises a binder. In some embodiments, the binder can comprise any of the following or combinations of the following materials: monosaccharides such as glucose, fructose, mannose, arabinose; di- and trisaccharides such as sucrose, lactose, maltose, trehalose, lactulose; corn and rice syrup solids; dextrins such a corn, wheat, rice and tapioca dextrins; maltodextrins; starches such as rice, wheat, corn, potato, tapioca starches, or these starches modified by chemical modification; alginates, chitosans; gums such as carrageen, and gum arabic; polyols such as glycerol, sorbitol, mannitol, xylitol, erythritol; esters of polyols such as sucrose esters, polyglycol esters, glycerol esters, polyglycerol esters, sorbitan esters; sorbitol; molasses; honey; gelatins; peptides; proteins and modified proteins such as whey liquid, whey powder, whey concentrate, whey isolate, whey protein isolate, high lactose whey by-product, meat broth solids such as chicken broth, chicken broth solids, soy protein, and egg white.

In some embodiments, lipids and lipid derivatives can also be used as binder components. Lipids can be used in combination with water and/or other binder components. Lipids can include plant fats such as soybean oil, corn oil, rapeseed oil, olive oil, safflower oil, palm oil, coconut oil, palm kernel oil, and partially and fully hydrogenated derivatives thereof; animal fats and partially and fully hydrogenated derivatives thereof; and waxes.

In some embodiments, the composition has a palatability equal to that of a control composition.

Other embodiments provide methods for: a) increasing the food intake of a companion animal suffering from stress; b) maintaining the food intake of a companion animal during a stressful experience; c) increasing and/or maintaining the food intake of a companion animal suffering from anxiety; d) normalizing the food intake of a companion animal during schedule variations; e) decreasing fecal ethanolamides in a companion animal; f) decreasing fecal acyltaurines in a companion animal; g) decreasing fecal acylglycines in a companion animal; h) decreasing dietary protein putrefaction; i) increasing the weight of a companion animal suffering from anxiety; and/or j) modulating the endocannabinoid system of a companion animal; comprising: administering a composition comprising an insoluble fiber source comprising a lignin; a polyphenol source; and a soluble fiber source; wherein the weight ratio of insoluble fiber to soluble fiber is greater than about 2:1, to a companion animal in need thereof.

Still further embodiments provide methods for: a) increasing the food intake of a companion animal suffering from stress; b) maintaining the food intake of a companion animal during a stressful experience; c) increasing and/or maintaining the food intake of a companion animal suffering from anxiety; d) normalizing the food intake of a companion animal during schedule variations; e) decreasing fecal ethanolamides in a companion animal; f) decreasing fecal acyltaurines in a companion animal; g) decreasing fecal acylglycines in a companion animal; h) decreasing dietary protein putrefaction; i) increasing the weight of a companion animal suffering from anxiety; and/or j) modulating the endocannabinoid system of a companion animal; comprising: administering any one of the compositions described herein to companion animal in need thereof.

Yet other embodiments of the present invention provide the use of any one of the compositions described herein in the manufacture of a pet food composition for: a) increasing the food intake of a companion animal suffering from stress; b) maintaining the food intake of a companion animal during a stressful experience; c) increasing and/or maintaining the food intake of a companion animal suffering from anxiety; d) normalizing the food intake of a companion animal during schedule variations; e) decreasing fecal ethanolamides in a companion animal; f) decreasing fecal acyltaurines in a companion animal; g) decreasing fecal acylglycines in a companion animal; h) decreasing dietary protein putrefaction; i) increasing the weight of a companion animal suffering from anxiety; and/or j) modulating the endocannabinoid system of a companion animal.

As used herein, the term “a companion animal” refers to an animal of any species kept by a caregiver as a pet or any animal of a variety of species that have been widely domesticated as pets, including dogs (Canis familiaris) and cats (Felis domesticus), whether or not the individual animal is kept solely or partly for companionship. Thus, companion animals include, for example and not limitation, working dogs, farm cats kept for rodent control, pet cats, pet dogs, ferrets, birds, reptiles, rabbits, and fish. In some embodiments, the companion animal is selected from a canine and a feline. In other embodiments, the companion animal is a feline.

Companion animals exposed to stressful environments or schedule variations may present with one of more of the following clinical, diagnostic or behavioral findings:

Clinical
Cardiovascular
   Tachycardia, bradycardia, hypertension (e.g. “white-coat hypertension”)
Increased respiratory rate
Dilated pupils
Hyperthermia
Gastrointestinal
   Upper gastrointestinal tract
      Expulsion of hair, food, or bile from the mouth
   Lower gastrointestinal tract
      Diarrhea, constipation, soft stool that is blood tinged and covered with mucous (“stress colitis”), evacuation of anal sacs
Lower urinary tract
   Stranguria, hematuria, or pollakiuria
Skin
   Epilation, skin lesions, or chin acne

Diagnostic
Complete blood count
   Platelet hypersensitivity
   Lymphocytosis
   Neutrophilia
Blood chemistry tests
   Elevated cortisol
   Hyperglycemia
   Hypokalemia
Urinalysis
   Elevated urine pH

Behavioral
Visual presentation
   Enlarged pupils, downward/backward facing ears, arched back
Anorexia or decreased food and water intake
Avoidance behaviors
   Lethargy, somnolence, withdrawal, guarding, reduced activity level, and decreased social interactions with conspecifics and caretakers
Aggressive, hypervigilant, hiding behaviors
Urination and defecation outside the litter box (separate from other signs referable to the lower urinary and gastrointestinal tracts)
Lethargy, somnolence, enhanced pain-like behaviors, and decreased general activity, body-care activities (grooming), and social interactions

As used herein, the term “soluble fiber” means one or more fibers that are readily fermented in the large intestine, e.g., beet pulp, guar gum, chicory root, psyllium, pectin, blueberry, cranberry, squash, apples, oats, beans, citrus, or barley.

Insoluble fiber may be supplied by any of a variety of sources, including cellulose, whole wheat products, wheat oat, corn bran, flax seed, grapes, celery, green beans, cauliflower, potato skins, fruit skins, vegetable skins, peanut hulls, and soy fiber.

In instances when the composition is an animal's food, vitamins and minerals can be included in amounts required to avoid deficiency and maintain health. These amounts are readily available in the art. The Association of American Feed Control Officials (AAFCO) provides recommended amounts of such ingredients for dogs and cats. See Association of American Feed Control Officials. Official Publication, pp. 126-140 (2003). Vitamins useful as food additives include, e.g., vitamin A, vitamin B₁, vitamin B₂, vitamin B₆, vitamin B₁₂, vitamin C, vitamin D, vitamin E, vitamin H (biotin), vitamin K, folic acid, inositol, niacin, and pantothenic acid. Minerals and trace elements useful as food additives include calcium, phosphorus, sodium, potassium, magnesium, copper, zinc, choline, and iron salts.

The compositions of the present invention may further contain additives known in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may increase the shelf life of the composition. Suitable examples can include preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches.

Additives for coloring, palatability, and nutritional purposes can include colorants, salts (including but not limited to sodium chloride, potassium citrate, potassium chloride, and other edible salts), vitamins, minerals, and flavoring. The amount of such additives in a composition typically is up to about 5% by weight (on a dry matter basis of the composition). Other additives can include antioxidants, omega-3 fatty acids, omega-6 fatty acids, glucosamine, chondroitin sulfate, vegetable extracts, herbal extracts, etc.

The term “palatability”, as used herein, encompasses all of the various properties of the food sensed by the animal, such as texture, taste and aroma.

Some embodiments of the present invention comprise a carbohydrate source. The carbohydrate source can comprise cereals, grains, corn, wheat, rice, oats, corn grits, sorghum, grain sorghum/milo, wheat bran, oat bran, amaranth, Durum, and/or semolina.

Some embodiments of the present invention comprise a fat source. The fat source, or fat ingredient, can comprise poultry fat, chicken fat, turkey fat, pork fat, lard, tallow, beef fat, vegetable oils, corn oil, soy oil, cottonseed oil, palm oil, palm kernel oil, linseed oil, canola oil, rapeseed oil, fish oil, menhaden oil, anchovy oil, and/or olestra.

Other embodiments of the present invention comprise additional ingredients. For example, these additional ingredients can include active ingredients, such as sources of fiber, minerals, vitamins, amino acids, carotenoids, antioxidants, fatty acids, glucose mimetics, probiotics, prebiotics, and others.

Sources of fiber can include, for example, fructooligosaccharides (FOS), beet pulp, mannanoligosaccharides (MOS), oat fiber, citrus pulp, carboxymethylcellulose (CMC), guar gum, gum arabic, apple pomace, citrus fiber, fiber extracts, fiber derivatives, dried beet fiber (sugar removed), cellulose, a-cellulose, galactooligosaccharides, xylooligosaccharides, and oligo derivatives from starch, inulin, psyllium, pectins, citrus pectin, guar gum, xanthan gum, alginates, gum arabic, gum talha, beta-glucans, chitins, lignin, celluloses, non-starch polysaccharides, carrageenan, reduced starch, soy oligosaccharides, trehalose, raffinose, stachyose, lactulose, polydextrose, oligodextran, gentioligosaccharide, pectic oligosaccharide, and/or hemicellulose.

Mineral sources can include, for example, sodium selenite, monosodium phosphate, calcium carbonate, potassium chloride, ferrous sulfate, zinc oxide, manganese sulfate, copper sulfate, manganous oxide, potassium iodide, and/or cobalt carbonate. In some embodiments, pecan shells may also be a source of lignin-based fiber.

Suitable vitamins may include choline chloride, vitamin E, ascorbic acid, vitamin A acetate, calcium pantothenate, pantothenic acid, biotin, thiamine mononitrate (source of vitamin B1), vitamin B12 supplement, niacin, riboflavin supplement (source of vitamin B2), inositol, pyridoxine hydrochloride (source of vitamin B6), vitamin D3 supplement, folic acid, vitamin C, and/or ascorbic acid.

Sources of polyphenols ingredients can include tea extract, rosemary extract, rosemarinic acid, coffee extract, pecan shells, caffeic acid, turmeric extract, blueberry extract, grape extract, grapeseed extract, and/or soy extract.

Sources of amino acids can include 1-Tryptophan, Taurine, Histidine, Carnosine, Alanine, Cysteine, Arginine, Methionine, Tryptophan, Lysine, Asparagine, Aspartic acid, Phenylalanine, Valine, Threonine, Isoleucine, Histidine, Leucine, Glycine, Glutamine, Taurine, Tyrosine, Homocysteine, Ornithine, Citruline, Glutamic acid, Proline, and/or Serine.

Sources of carotenoids may include lutein, astaxanthin, zeaxanthin, bixin, lycopene, and/or beta-carotene. Sources of antioxidant ingredients can include tocopherols (vitamin E), vitamin C, vitamin A, plant-derived materials, carotenoids (described above), selenium, and/or CoQ10 (Co-enzyme Q10). Sources of fatty acid ingredients can include arachidonic acid, alpha-linoleic acid, gamma linolenic acid, linoleic acid, eicosapentanoic acid (EPA), docosahexanoic acid (DHA), and/or fish oils as a source of EPA and/or DHA.

Sources of glucose mimetics can include glucose anti-metabolites including 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and/or avocado extract comprising mannoheptulose.

Still other ingredients may include beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, L-carnitine, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), anti-foaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, and/or keratolytics.

Suitable probiotics may include one or more bacterial probiotic microorganisms suitable for pet consumption and effective for improving the microbial balance in the pet gastrointestinal tract or for other benefits, such as disease or condition relief or prophylaxis, to the pet. Various probiotic microorganisms known in the art. In some embodiments, the probiotic component may be selected from bacteria, yeast or microorganism of the genera Bacillus, Bacteroides, Bifidobacterium, Enterococcus (e.g., Enterococcus faecium DSM 10663 and Enterococcus faecium SF68), Lactobacillus, Leuconostroc, Saccharomyces, Candida, Streptococcus, and mixtures of any thereof. In other embodiments, the probiotic may be selected from the genera Bifidobacterium, Lactobacillus, and combinations thereof. Those of the genera Bacillus may form spores. In further embodiments, the probiotic does not form a spore. Non-limiting examples of lactic acid bacteria suitable for use herein include strains of Streptococcus lactis, Streptococcus cremoris, Streptococcus diacetylactis, Streptococcus thermophilus, Lactobacillus bulgaricus, Lactobacillus acidophilus (e.g., Lactobacillus acidophilus strain DSM 13241), Lactobacillus helveticus, Lactobacillus bifidus, Lactobacillus casei, Lactobacillus lactis, Lactobacillus plantarum, Lactobacillus rhamnosus, Lactobacillus delbrukii, Lactobacillus thermophilus, Lactobacillus fermentii, Lactobacillus salvarius, Lactobacillus reuteri, Bifidobacterium longum, Bifidobacterium infantis, Bifidobacterium bifidum, Bifidobacterium animalis, Bifidobacterium pseudolongum, and Pediococcus cerevisiae, or mixtures of any thereof. In specific embodiments, the probiotic-enriched coating may comprise the bacterial strain Bifidobacterium animalis AHC7 NCIMB 41199.

As used herein, the term “kibble” includes a particulate pellet like component of animal feeds, such as dog and cat feeds, typically having a moisture, or water, content of less than 12% by weight. Kibbles may range in texture from hard to soft. Kibbles may range in internal structure from expanded to dense. Kibbles may be formed by an extrusion process. In non- limiting examples, a kibble can be formed from a core and a coating to form a kibble that is coated, also called a coated kibble. It should be understood that when the term “kibble” is used, it can refer to an uncoated kibble or a coated kibble.

Some embodiments of the present invention comprise a protein source. The protein source, or protein ingredient, can comprise chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grains solubles and single-cell proteins, for example yeast, algae, and/or bacteria cultures.

In some embodiments, the pet food compositions may be prepared in a non-kibble format, for example, as a loaf, a stew, a “meat and gravy” form, a gruel, shreds with a moisture content greater than 50%”, or as a product that could be pushed through a syringe.

Embodiments of the present invention will now be further described by way of the following, non-limiting, examples.

### EXAMPLES

Diets are formulated according to AAFCO (American Association of Feed Control Officials) and NRC (National Research Council) nutrition recommendation. Finished kibble is produced by extrusion, dried and coated with palatants. To prepare an exemplary composition of the present invention (Example 1, or Ex. 1), ingredients are added to the grain mix prior to hammer-mill homogenization and extrusion. Example I contains a selection of fibers including lignin, and also excludes higher levels of added soluble fiber. In contrast, the comparative diet (Comparative Example 1, or Comp. Ex. 1) contains the same nutritional components as Ex. 1, but a higher added soluble fiber content and a lower insoluble fiber content. An exemplary composition of the present invention and the comparative composition are described below in Table 1.

TABLE 1

| | |
|---|---|
| Ex. 1 | Comp. Ex. 1 |

| | |
|---|---|
| Ingredients | Wt. % |

| | | |
|---|---|---|
| Corn, gluten, meal | 24.69 | 23.05 |
| Rice, brown | 17.94 | 19.57 |
| Chicken dried 10% ash | 11 | 11 |
| Poultry by-product meal | 10 | 10 |
| Pork Fat, Choice White Grease | 8 | 8 |
| Polyphenol Source | 8 | 8 |
| Insoluble Fiber | 10 | — |
| Chicken liver digest | 2 | 2 |
| Calcium sulfate | 1.2 | 1.2 |
| Lactic acid, blend 84% | 1.2 | 1.2 |
| Fish oil, TG | 0.78 | 0.78 |
| Potassium chloride | 0.66 | 0.66 |
| Soybean oil | 0.5 | 0.5 |
| Sodium chloride | 0.45 | 0.45 |
| Choline chloride | 0.45 | 0.45 |
| Vitamin E, oil, 29% | 0.43 | 0.43 |
| Amino Acid(s) | 0.47 | 0.47 |
| Vitamin and Mineral Premix | 0.23 | 0.23 |
| Flax seed | 2 | 3 |
| Soluble Fiber | — | 9 |

The compositions described in Table 1 (above) may be prepared using conventional means known to those skilled in the art.

An Institutional Animal Care and Use Committee (IACUC) approved clinical dietary intervention protocol is implemented which enrolled healthy feline subjects randomized to two groups based on age, weight and sex. Cats are assessed as healthy by markers of biochemical and clinical health. The study is a caretaker-blinded, longitudinal design with cats fed experimental foods for 91 days. Both diets (Ex. 1 and Comp. Ex. 1) retain the same overt nutritional qualities while varying in their inclusion of specialty experimental ingredients that affected soluble-, insoluble-, crude- and total- dietary fiber levels. Specifically, Ex. 1 was designed to have reduced soluble fiber and increased insoluble fiber, as compared to Comp. Ex. 1.

Diet intakes are monitored as grams consumed by difference from starting bowl weight and then converted to calories before normalizing to metabolic body weight (kcal/kg∧0.75). Feces and serum metabolites are collected at the beginning and end of study, values for metabolites are natural-log transformed and used to calculate differences (deltas from the beginning to end of study) when consuming either Comp. Ex. 1 or Ex. 1. Statistical analyses are performed with JMP Software v13.1 (SAS Institute, NC). Unless otherwise noted, univariate tests are performed by t-test on deltas (end of study—beginning of study) of natural-log transformed metabolite levels with alpha as 0.05. Multivariate tests for metabolite classes are performed by MANOVA with contrast at alpha=0.05.

During this trial, compared to Comp. Ex. 1, an exemplary composition of the present invention significantly increases intake of calories normalized to metabolic body-weight, the gold-standard for measuring intake differences that accounts for individual differences in metabolism based on bodyweight. These results are described below in Table 2.

TABLE 2

| | Ex. 1 | Comp. Ex. 1 |
|---|---|---|
| Average Caloric Intake | 70 | 63 |

Schedule variations are a normal part of care and maintenance of pet health, and when pets are group housed in a social environment they often experience schedule variations as a group. This allows summary and analysis trends which occur in individual households and with individual pets. In particular, shifting between housing units for routine maintenance or visits to the vet for health screening in the research setting serves to mimic a pet parent boarding their pet or having a regular health checkup for their pet, respectively.

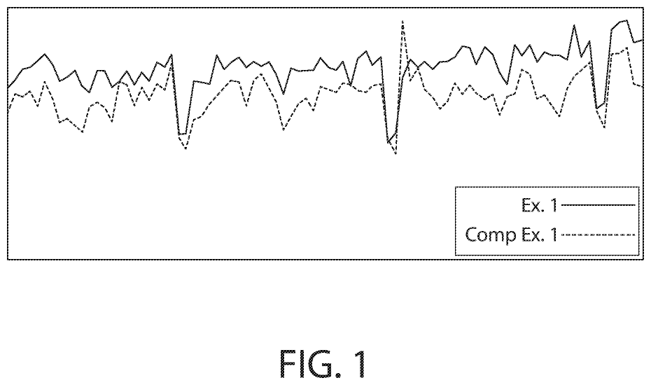

It is apparent from analysis of trends that intakes of cats over the course of the 91-day study are periodically perturbed by regular processes. Also apparent is that compositions of the present invention support a more regular intake pattern with fewer perturbations and a quicker return to normalcy, than comparative compositions that do not contain the inventive combination of ingredients. (See, FIG. 1).

### Example 2

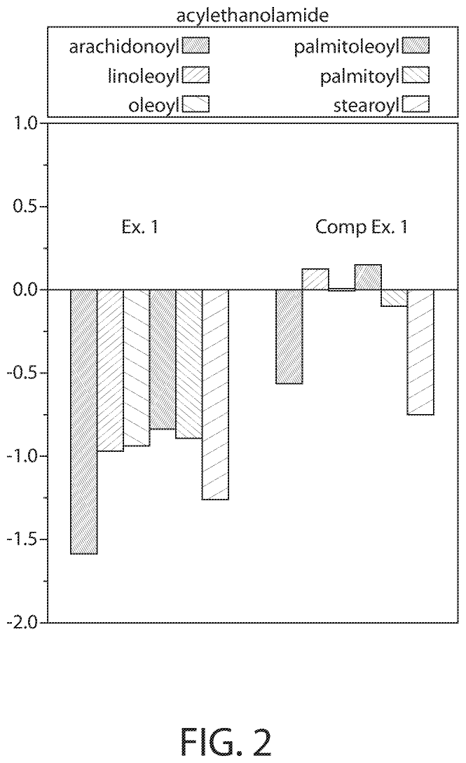

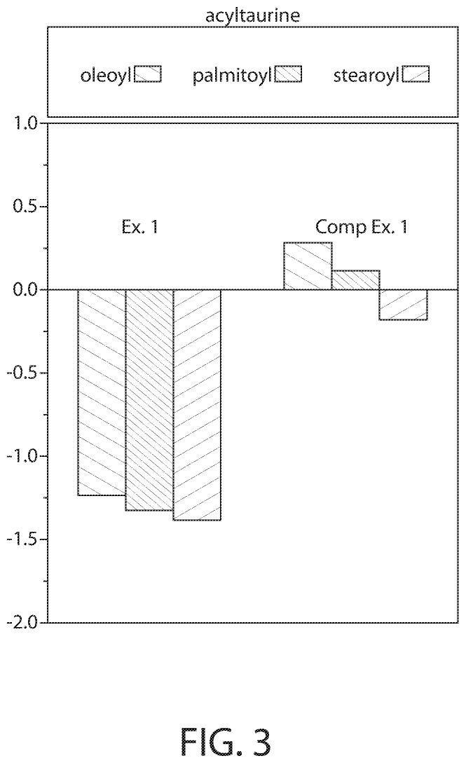

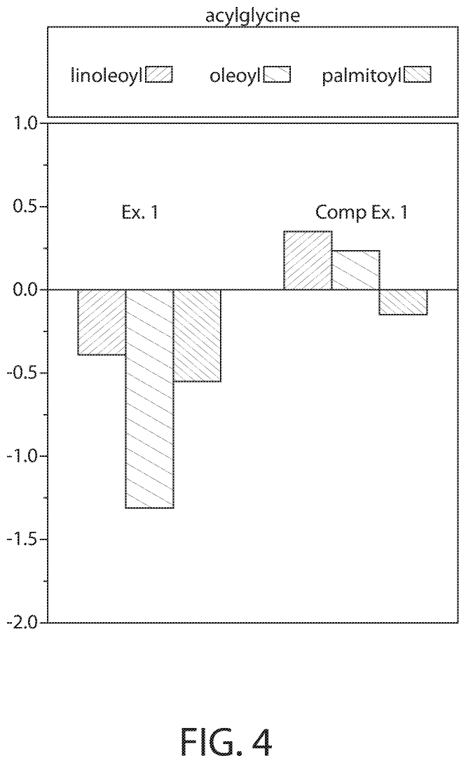

N-Acylethanolamides (e.g. oleoylethanolamide) and other N-acylamines (NAA) are strong drivers of food intake and satiety that are active locally in the intestine. It has recently been recognized that these bioactive signaling hormones are also produced by the gut microbiota. Thus, the gut microbiota produce metabolic products (called postbiotics) which determine host physiological processes of food intake and satiety. An exemplary composition of the present invention strongly and uniformly decreases nearly all members of several classes of NAA (see, FIGS. 2-4): ethanolamides (FIG. 2); taurines (FIG. 3) and glycines (FIG. 4).

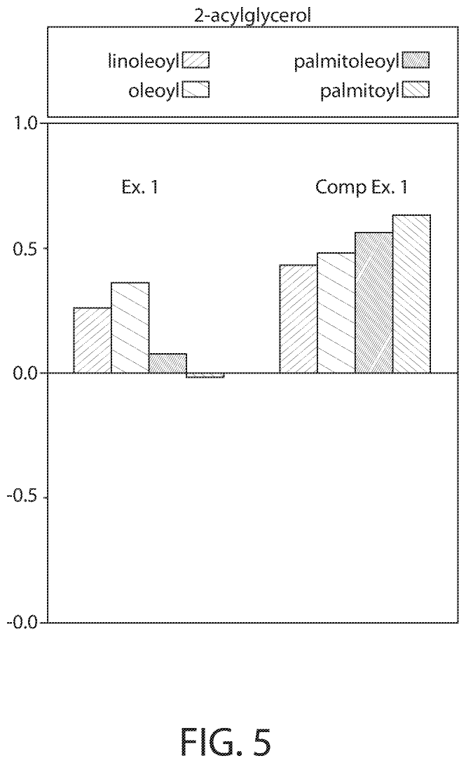

Surprisingly, the O-linked O-acylglycerol class (OAG) are relatively unchanged by an exemplary composition of the present invention (see, FIG. 5), indicating the profound specificity (targeted efficacy) of an exemplary composition of the present invention. These results indicate that compositions of the present invention are expected to reduce gastrointestinal satiety hormones and increase food intake, as well as normalize intakes during schedule variations.

### Example 3

When dietary protein is inadequately digested and absorbed, the bypass nitrogen is putrefied in the colon by proteolytic microbiota. Two types of microbial metabolic products of undigested protein are polyamines (which contain the nitrogen fraction of the protein) and branched short chain fatty acids (BSCFA), which contain the carbon skeleton of the undigested protein. Decreases in these products, especially when paired with decreased levels in serum circulation, can show that dietary protein is being more appropriately digested and absorbed, with less occurrence of protein putrefaction. The double benefit is that protein is used more efficiently by the pet directly, and concurrently, the microbes are not producing toxic byproducts of protein putrefaction.

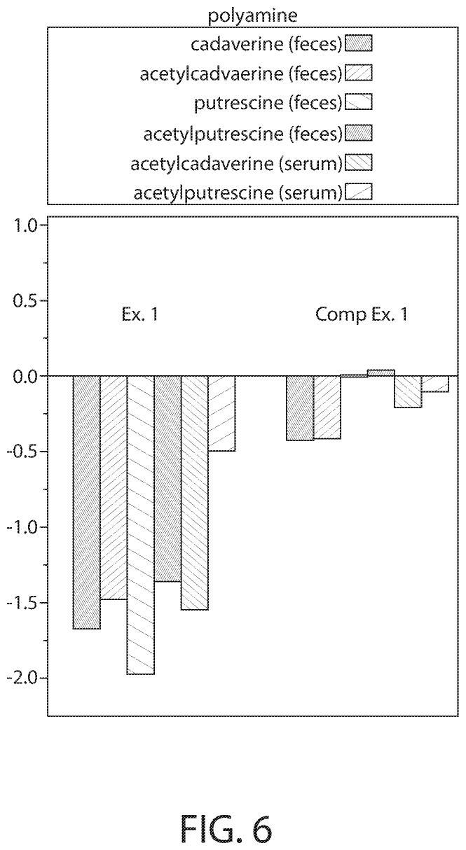

As illustrated by the data described in FIG. 6, an exemplary composition of the present invention improves digestion and metabolic disposal of dietary protein, as indicated by reduced levels of cadaverine-type and putrescine-type polyamines in the feces of invention-fed cats. These polyamines stem from putrefaction of lysine and arginine amino acids, respectively. This reduction in the gastrointestinal compartment has a systemic effect, as demonstrated by the reduction in serum polyamines shown in FIG. 6.

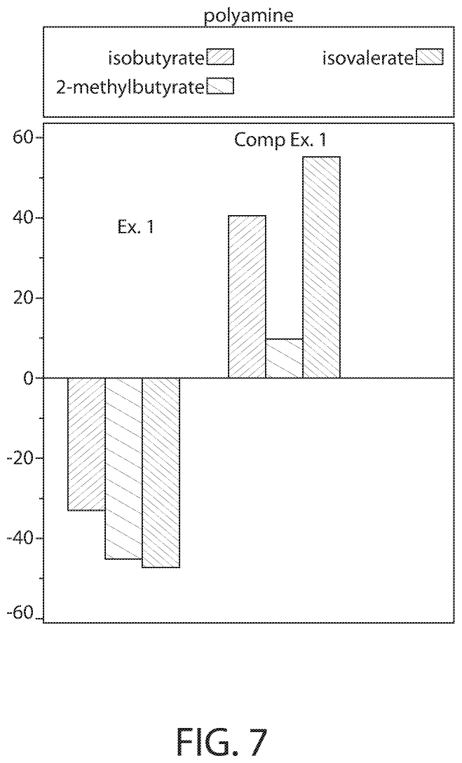

Fecal levels of BSCFA are also decreased (see, e.g., FIG. 7), confirming the reduction in protein putrefaction shown with polyamines. All three canonical BSCFA, derived from valine, leucine and isoleuce putrefaction respectively, are significantly decreased relative to the cats fed with the comparative composition (Comp. Ex. 1).

As demonstrated by the foregoing Examples, including the data described in Table 2 and FIGS. 1 to 7, a diet based on compositions of the present invention—containing controlled-high levels of insoluble fiber, especially lignin, and controlled-low levels of added soluble fiber—increases overall food intake and expedites a return to ‘normal’ intakes when the animal undergoes a schedule variation. This was an unexpected outcome, as lignin is an indigestible fiber and fiber inclusion typically decreases dietary intakes.

In summary, compositions of the present invention have demonstrated an ability to increase food intake, maintain protein digestibility, and decrease markers of dietary protein putrefaction, which can play a critical role in improving pet health in the face of schedule variation.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A pet food composition comprising:
an insoluble fiber source comprising a lignin;
a polyphenol source; and
a soluble fiber source;
wherein the weight ratio of insoluble fiber to soluble fiber is greater than about 2:1.

2. The pet food composition according to claim 1, wherein the polyphenol source comprises a mixture of phenolic compounds.

3. The pet food composition according to claim 1, wherein the polyphenol source comprises a phenolic compound selected from ellagic acid; gallic acid;
protocatechuic acid; p-hydroxybenzoic acid; catechin; and a combination of two or more thereof.

4. The pet food composition according to claim 1, wherein the insoluble fiber source comprises a fiber bundle.

5. The pet food composition according to claim 4, wherein the fiber bundle comprises a mixture of at least two different sources of insoluble fiber.

6. The pet food composition according to claim 4, wherein the fiber bundle comprises a cellulosic material; tomato pomace and a natural vegetable food fiber.

7. The pet food composition according to claim 4, wherein the natural vegetable food fiber is pea fiber.

8. The pet food composition according to claim 1, wherein the polyphenol source comprises pecan shells.

9-10. (canceled)

11. The pet food composition according to claim 1, wherein the weight ratio of insoluble fiber to soluble fiber is about 10:1.

12. (canceled)

13. A composition for stimulating food intake in a companion animal in need thereof, comprising:
a source of soluble fiber;
a complex comprising:
a source of polyphenols; and
a source of insoluble fiber comprising a fiber bundle;
wherein the total dietary fiber content of the composition is greater than 5 percent on a dry matter basis (DMB).

14. The composition according to claim 13, wherein the weight ratio of insoluble fiber to soluble fiber is greater than about 3:1.

15. The composition according to claim 13, wherein the source of soluble fiber comprises less than about 1%, by weight, of the total composition.

16. The composition according to claim 13, wherein the fiber bundle comprises a mixture of at least two different sources of insoluble fiber.

17. (canceled)

18. The composition according to claim 13, wherein the fiber bundle comprises from about 5% to about 15%, by weight, of the total composition.

19. (canceled)

20. The composition according to claim 13, wherein the polyphenol source comprises a phenolic compound selected from ellagic acid; gallic acid; protocatechuic acid; p-hydroxybenzoic acid; catechin; and a combination of two or more thereof.

21. The composition according to claim 13, wherein the polyphenol source comprises pecan shells.

22. (canceled)

23. The composition according to claim 13, wherein the weight ratio of insoluble fiber to soluble fiber is greater than about 7.5:1.

24-25. (canceled)

26. The composition according to claim 13, wherein the food intake is calculated based upon intake of calories normalized to metabolic bodyweight.

27-40. (canceled)

41. The composition according to claim 13, wherein the companion animal is selected from a canine and a feline.

42-43. (canceled)

44. A method for:
a) increasing the food intake of a companion animal suffering from stress;
b) maintaining the food intake of a companion animal during a stressful experience;
c) increasing and/or maintaining the food intake of a companion animal suffering from anxiety;
d) normalizing the food intake of a companion animal during schedule variations;
e) decreasing fecal ethanolamides in a companion animal;
f) decreasing fecal acyltaurines in a companion animal;
g) decreasing fecal acylglycines in a companion animal;
h) decreasing dietary protein putrefaction;
i) increasing the weight of a companion animal suffering from anxiety;
j) maintaining levels of acylglycerols; and/or
k) modulating the endocannabinoid system of a companion animal;
comprising:
administering a composition comprising
an insoluble fiber source comprising a lignin;
a polyphenol source; and
a soluble fiber source;
wherein the weight ratio of insoluble fiber to soluble fiber is greater than about 2:1, to a companion animal in need thereof.

45-51. (canceled)
